(19) United States
(12) Patent Application Publication
(10) Pub. No.: US 2019/0090728 A1
(43) Pub. Date: Mar. 28, 2019

(54) VISUALIZATION SYSTEM COMPRISING AN OBSERVATION APPARATUS AND AN ENDOSCOPE

(71) Applicant: Carl Zeiss Meditec AG, Jena (DE)

(72) Inventors: Martin Fanenbruck, Oberkochen (DE); Helge Jess, Oberkochen (DE); Roland Guckler, Ulm (DE)

(21) Appl. No.: 16/139,032

(22) Filed: Sep. 22, 2018

(30) Foreign Application Priority Data
Sep. 22, 2017 (DE) 10 2017 216 853.6
Nov. 6, 2017 (DE) 10 2017 219 621.1

(51) Int. Cl. A61B 1/05; A61B 34/20; G02B 21/00; H04N 5/247

(52) U.S. Cl. CPC A61B 1/05; A61B 34/20; G02B 21/0012; H04N 2005/2255; A61B 2562/0219; A61B 2034/2051; H04N 5/247

(57) ABSTRACT

A visualization system includes an observation apparatus having a first image recording device to observe an operation region with a first observation plane, and an endoscope having a probe and a second image recording device to observe the operation region with a second observation plane. A display device represents a first image recorded by the first image recording device in a first orientation and a second image recorded by the second image recording device in a second orientation. The endoscope includes a motion sensor connected to a control unit to determine an angular position of the probe of the endoscope in space. The control unit determines an angular position of the probe of the endoscope relative to a first viewing axis to permit the second orientation of the second image to be alignable depending on an angular position of the probe relative to the first viewing axis.

### CROSS REFERENCE TO RELATED APPLICATIONS

This application claims priority to German patent application DE 10 2017 216 853.6, filed Sep. 22, 2017, and to German patent application DE 10 2017 219 621.1, filed on Nov. 6, 2017, the entire contents of which are incorporated herein by reference.

### TECHNICAL FIELD

The invention relates to a visualization system comprising an observation apparatus and an endoscope.

### BACKGROUND

An endoscope is a visualization instrument that is used during an examination or during an operation on a patient. An endoscope comprises a probe that can be introduced into body channels, in particular into narrow and deep operation channels or cavities, in order to be able to view anatomical structures or body tissue of an operation region. One particular field of use is neurosurgery.

An endoscope is a manually guided medical instrument and can be used in addition to the observation apparatus in different positions in order to look at structures that are hidden in the microscopic view. The probe tip can have a mechanical marking in order to indicate a viewing direction of the probe. As soon as the probe tip is hidden by a tissue structure, however, the viewing direction of the probe is no longer discernible to the surgeon. When the image generated by the endoscope is viewed on a display device, the coordination of the direction of movement of the probe by a user's hand, i.e., the hand-eye coordination, is hampered if the viewing direction of the probe tip is not clearly discernible.

### SUMMARY

Therefore, it is an object of the invention to provide a visualization system comprising an observation apparatus and an endoscope in which the alignment of a probe of the endoscope is discernible and the hand-eye coordination during the movement of the endoscope is improved.

The object is achieved by a visualization system comprising an observation apparatus and an endoscope as disclosed herein.

According to an aspect of the invention, a visualization system comprises an observation apparatus having a first image recording device for observing an operation region with a first observation plane, wherein in the first observation plane, a viewing direction is defined by a first viewing axis Y1.

The visualization system comprises an endoscope having a probe and a second image recording device configured to observe the operation region with a second observation plane with a second viewing axis Y2.

The visualization system comprises a display device, which represents a first image recorded by the first image recording device in a first orientation and a second image recorded by the second image recording device in a second orientation. A control unit is connected to the second image recording device and the display device.

The endoscope comprises a motion sensor, which is connected to the control unit, an angular position of the probe of the endoscope in space being determinable by said motion sensor. The control unit is configured to the effect that an angular position of the probe of the endoscope relative to the first viewing axis Y1 is determinable by evaluation of the data of the motion sensor, such that the second orientation of the second image is alignable depending on an angular position of the probe relative to the first viewing axis Y1.

The visualization system comprises an observation apparatus having a first image recording device and an endoscope having a second image recording device, and also a display device and a control unit.

The observation apparatus is configured to observe an operation region in a first observation plane, wherein in the first observation plane, a viewing direction is defined by a first viewing axis Y1.

The observation apparatus can be an optical surgical microscope comprising eyepieces and one or more cameras. The observation apparatus can also be formed by a digital image capture system comprising a camera and an optical unit. The surgical microscope can also be formed only by a camera.

The operation region is a tissue region to be operated on, which is also referred to as operation site. A viewing direction is a direction of view of an observer viewing an observation plane. A viewing axis is a reference axis that defines the direction of view of the observer relative to the observation plane. Said reference axis can also be referred to as “0°” axis. Relative to a coordinate system of the first observation plane that is defined by the orthogonal axes X1, Y1 and Z1, the first viewing axis is defined by the axis Y1. A first viewing direction defines the direction of view with respect to the first observation plane.

In this case, the observable region of the operation site is not restricted to the first observation plane. The observable operation region is a three-dimensional region. The first observation plane defines a plane that is defined by the observation optical unit of the observation apparatus. The observation optical unit of the observation apparatus can also sharply image a region above and below the first observation plane, said region being defined by the depth of focus.

The operation region is recorded by the first image recording device and displayed in a first image in a first orientation on the display device. The first image represented on the display device can be an individual image, a sequence of individual images at specific points in time or a video image, also in real time.

The orientation of an image defines the alignment of a displayed image on the display device at a specific rotation angle. To that end, the first image recorded by the first image recording device can be rotated on the display device by an angle perpendicular to the first observation plane, about the Z1 axis, in such a way that a specific region is arranged at the top on the display device. The first image can be displayed in a first orientation on the display device in such a way that that region of the image which lies on the positive side on the first viewing axis Y1 is arranged at the top. If an observer looks along the direction of the first viewing axis Y1, the image recorded by the first image recording device can be displayed on the display device directly, without a change in the first orientation, i.e., without rotation angle correction.

The endoscope comprises a probe that is arranged on a handpiece and is guided manually by an observer. A probe is a thin tube several centimeters in length which can be introduced into a tissue region or a tissue structure. The image captured at the probe tip, the distal end of the probe, is guided via optical waveguides to the second image recording device. The operation region observable by the probe in a second observation plane is captured by the second image recording device and represented as a second image in a second orientation on the display device. The second image can be an individual image, a sequence of individual images at specific points in time, or a video image.

The first observation plane and the second observation plane are different observation planes. These two observation planes can be arranged at an angle with respect to one another. The first image and the second image show different views of the operation region. The first image and the second image can each comprise individual images and/or video image sequences.

A control unit is connected to the second image recording device and the display device. The second image recording device of the endoscope is connected to the display device via the control unit, such that the recorded images can be computationally processed, rotated and/or altered. For this purpose, the control unit can comprise an image processing unit. The control unit comprises information about the alignment of the first viewing axis Y1. This information can be stored as a fixed numerical value in the control device.

The control unit processes the images of the second image recording device and determines the position of the second viewing axis Y2 therefrom. The second viewing axis Y2 is a reference axis that defines a direction of view of the probe relative to the tissue region viewed in a second observation plane. The second viewing axis Y2 can be defined by the geometric and optical construction of the endoscope. The second viewing axis Y2 can lie geometrically in the plane spanned by a center axis of the probe and of the handpiece of the probe. The second viewing axis Y2 can be identical to a mechanical marking of the probe tip, for example a jumper. The inserted second viewing axis Y2 can also be manually adapted to an observer. By way of example, an observer who guides the endoscope using the left hand may have the need to indicate the second viewing axis Y2 subjectively in a different second orientation than an observer who guides the endoscope using the right hand. The observer can set the image to the observer's movement coordination by rotating the second viewing axis Y2 into a second orientation.

The endoscope comprises a motion sensor, which is connected to the control unit, an angular position of the probe of the endoscope in space being determinable by said motion sensor. The motion sensor is configured to capture a movement of the endoscope and to generate an electronically evaluatable movement value that can be evaluated by the control unit. A movement is characterized for example by a position change and/or an angular change of the endoscope in space. A movement can be uniform or comprise an acceleration. A movement can also be detected if it proves to be very small.

A motion sensor can capture a position change and/or an angular change in space. To that end, a motion sensor can for example be configured as a position sensor and determine an absolute angular position in space or determine a relative angular change with respect to a known angular position in space. As a result, an angular position of the probe in space is capturable. The angular position defines a rotation angle about one, two or three spatial axes, independently of the absolute 3D spatial coordinates.

The control unit is configured to the effect that an angular position of the probe of the endoscope relative to the first viewing axis Y1 is determinable by evaluation of the data of the motion sensor, such that the second orientation of the second image is alignable depending on an angular position of the probe relative to the first viewing axis Y1.

Once the probe of the endoscope has been introduced into a tissue region, the probe tip is no longer visible to the user. The display device displays the image recorded by the second image recording device as a second image in a second orientation. The second orientation of the second image can be aligned in such a way that the second viewing axis Y2 is aligned in a relative position with respect to the first viewing axis Y1, said relative position being predefined by the control unit or the observer. The orientation of the second image with the second viewing axis Y2 of the endoscope can be adapted to the first orientation of the first viewing axis Y1 of the observation apparatus.

Upon a rotation of the probe about an axis, for example the longitudinal axis, without a tracking of the orientation of the second image, the second image would likewise be rotated on the display device.

The motion sensor arranged in the endoscope registers a movement of the endoscope. As a result of the angular position being determined by the motion sensor, the alignment of the probe with respect to the first viewing axis Y1 and with respect to the operation site is firstly captured and the alignment of the orientation of the second image is adapted. Upon a change in the position of the endoscope, the orientation of the second image can thus be tracked automatically. Consequently, an intuitive hand-eye coordination is advantageously possible for the observer who is manually guiding the endoscope.

Upon an alignment of the orientation of the second image with respect to the first viewing axis Y1, the second image is rotated on the display device in such a way that a direction of movement of the endoscope, for example in the direction of the first viewing axis Y1 of the microscope, is displayed as a movement on the display device in the second image in the same orientation as in the first image. The second orientation of the second image is alignable depending on an angular position of the probe relative to the first viewing axis Y1 and is trackable depending on the data of the motion sensor.

This shall be elucidated on the basis of an example. On a display device, the first image of the observation apparatus is oriented in such a way that the first viewing axis Y1 is displayed in a vertical direction. The probe of the endoscope is aligned in the direction of a surface normal with respect to the observation plane, but rotated by 30° relative to the center axis of the probe.

On the display device, without this alignment, the second image would likewise be rotated by 30° with respect to the vertical relative to the first image. Upon a movement of the endoscope parallel to the first viewing axis Y1 of the microscope, the direction of movement in the second image would run obliquely by 30° with respect to the vertical relative to the first image. The observer's hand-eye coordination would be made more difficult.

Upon an alignment of the second orientation of the second image relative to the first viewing axis Y1, the rotation angle of the second image on the display device is corrected by 30° relative to the first image. Consequently, upon a movement of the endoscope parallel to the first viewing axis Y1 of the microscope, the direction of movement in the second image is represented in the same direction as in the first image. The observer who manually drives the endoscope, perceives this movement in the second image likewise in the vertical direction. This facilitates the hand-eye coordination for the observer. As a result of the angular position being determined by a motion sensor, the second orientation of the second image can be aligned and tracked depending on an angular position.

By way of example, the rotation of the wrist, which rotation would lead to a rotation of the second image on the display device, can be compensated for by a detection of the rotation angle by the motion sensor and a computational compensation by the control unit. If the observer rotates the endoscope about the center axis of the probe, for example when changing the position of the endoscope, the second orientation of the second image remains constant on the display device. The tracking of the orientation of the second image makes it possible to maintain the hand-eye movement coordination.

In one exemplary embodiment of the invention, a graphical marking is inserted in the second image represented on the display device, said graphical marking indicating the direction of the second viewing axis Y2 in the second image, wherein the graphical marking is trackable in the second image depending on an angular position of the probe relative to the first viewing axis Y1.

Once the probe of the endoscope has been introduced into a tissue region, the probe tip is no longer visible to the user. In order to facilitate the handling of the endoscope for the user and to make the orientation of the probe tip of the endoscope discernible to the user, a graphical marking is inserted in the second image represented on the display device, said graphical marking indicating the direction of the second viewing axis Y2 in the second image. The control unit processes the images of the second image recording device and determines the position of the second viewing axis Y2 therefrom. The second viewing axis Y2 is inserted as a graphical marking into the second image represented on the display device. An alignment of the probe tip of the endoscope is thus discernible in the second image. The second image is displayed in a second orientation on the display device.

The control unit is configured to the effect that an angular position of the probe of the endoscope relative to the first viewing axis Y1 is determinable by evaluation of the data of the motion sensor, such that the graphical marking in the second image is trackable depending on an angular position of the probe relative to the first viewing axis Y1.

The display device displays the image recorded by the second image recording device as a second image together with the graphical marking. The graphical marking, indicating the second viewing axis Y2 of the endoscope, can be adapted to the first orientation of the first viewing axis Y1 of the observation apparatus. The observer who manually guides the endoscope can unambiguously assign the second viewing axis Y2 to the probe of the endoscope at any time by virtue of the marking in the second image.

Upon a rotation of the probe about an axis, for example the longitudinal axis, without a tracking, the graphical marking of the second viewing axis Y2 would likewise be rotated. The motion sensor arranged in the endoscope registers a movement of the endoscope. As a result of the angular position being determined by the motion sensor, the alignment of the probe with respect to the first viewing axis Y1 and with respect to the operation site is firstly captured and indicated by the graphical marking in the second image. Upon a change in the position of the endoscope, the graphical marking can thus be tracked automatically. Consequently, an intuitive hand-eye coordination is advantageously possible for the observer who is manually guiding the endoscope.

In one exemplary embodiment of the invention, the control unit is connected to the first image recording device.

In this case, the control unit is connected to the first image recording device, the second image recording device, and the display device. The first image recording device of the observation apparatus and the second image recording device of the endoscope are connected to the display device via the control unit, such that the recorded images can be computationally processed and altered. For this purpose, the control unit can comprise an image processing unit.

In one exemplary embodiment of the invention, the viewing direction of the endoscope is formed at an angle relative to the center axis of the probe of the endoscope.

In this way, it is possible to view a tissue region situated laterally with respect to the probe. This is advantageous if the probe is introduced in a narrow channel.

In one exemplary embodiment of the invention, the motion sensor is a sensor selected from a position sensor, an acceleration sensor, a vibration gyroscope sensor, and a gyrosensor.

All these sensors are cost-effective and available in miniaturized form.

In one exemplary embodiment of the invention, the motion sensor is a position sensor. The position sensor can determine an angular position in space. The position sensor is configured to determine a relative inclination angle with respect to a perpendicular axis. An angular position can thus be determined independently of an acceleration. Position sensors are cost-effective.

In one exemplary embodiment of the invention, the motion sensor is an acceleration sensor. An acceleration sensor is cost-effective and available in miniaturized form. Moreover, an acceleration sensor has a high measurement accuracy.

In one exemplary embodiment of the invention, the motion sensor is a vibration gyroscope sensor.

Simple position sensors may be restricted to one axial direction, such that movements that take place perpendicular to this axial direction cannot be detected. If a position sensor detects a perpendicular direction on the basis of the gravitational force, for example, a rotational movement perpendicular to the gravitational force direction cannot be detected. In the case of an endoscope, this may have the disadvantage that in the event of a specific alignment of the axis of the probe, for example in a perpendicular direction, a rotation about this axis cannot be perceived by the position sensor since no vertical component of the movement is present.

A vibration gyroscope sensor makes it possible to measure rotational movements. For this purpose, a vibration gyroscope sensor comprises at least one oscillatory system, for example a quartz oscillator. A vibration gyroscope sensor can comprise three quartz oscillators aligned orthogonally to one another. If a quartz oscillator is rotated perpendicular to the deflection direction α at the angular velocity ω, the Coriolis force F=dα/dt*ω acts perpendicular thereto on the oscillation system. The alteration can be detected by a piezoelectric pick-up, such that a rotational movement is determinable. Vibration gyroscope sensors can be made very small, for example on a microelectromechanical basis.

In one exemplary embodiment of the invention, the motion sensor is a gyrosensor.

A gyrosensor is a piezo-based acceleration or position sensor that can measure very small accelerations, rotational movements or position changes. Advantageously, the gyrosensor can simultaneously detect the acceleration value and the inclination angle. As a result, a single sensor can form an acceleration sensor and the position sensor. Gyrosensors can be made very small and are cost-effective.

In one exemplary embodiment of the invention, the motion sensor is arranged in the handpiece.

There is enough space for the sensor in the handpiece. Moreover, the sensor can be arranged on an electronics circuit board already present in the handpiece. This saves additional signal lines or power supply lines for the sensor.

In one exemplary embodiment of the invention, the handpiece comprises a position sensor and an acceleration sensor.

Advantageously, the two sensors can synergistically complement one another.

In one exemplary embodiment of the invention, the second image recording device is fixedly connected to the probe.

This is the mechanically simplest connection and thus cost-effective and compact. The endoscope can be calibrated in a simple manner.

In one exemplary embodiment of the invention, the second image recording device is arranged rotatably relative to the probe.

In this exemplary embodiment, the second image recording device is mounted rotatably relative to the optical unit of the probe. The recorded image can therefore be displayed directly on the display device. This reduces the computational complexity for image processing in the control unit and allows a faster image sequence on the display device.

In one exemplary embodiment of the invention, the control unit comprises an image processing unit.

An image processing unit can be formed for example by a specific computer chip or a graphics card that is optimized for fast image processing operations. It is thus possible to effect processing of the images and the insertion and/or tracking of the graphical marking particularly rapidly and in real time.

In one exemplary embodiment of the invention, at least two graphical markings are inserted in the second image on the display device.

In this way, two items of information can be made available to the user; by way of example, a first graphical marking can correspond to a mechanical marking of the probe tip and a second graphical marking can indicate a direction selectable by the user, or a center axis of the probe corresponding to a straight ahead view or advance direction of the probe. All graphical markings are trackable depending on the data of the motion sensor and thus on an angular position of the probe relative to the first viewing axis Y1.

In one exemplary embodiment of the invention, the alignment of the probe relative to the first observation plane is determinable by image evaluation of the images captured by the first image recording device.

At least one part of the probe is visible in the image captured by the first image recording device of the observation apparatus. The observation apparatus image is evaluatable by the control unit. An alignment of the probe relative to the first observation plane is thus determinable by evaluation of the image information of the first image recording device. This information about the alignment of the probe can be supplemented by the items of information provided by the motion sensor. The system can be calibrated on the basis of this information. Typically, the alignment of the probe relative to the first observation plane is already determinable before the first determination of an angular position by the motion sensor by image evaluation of the image captured by the first image recording device.

In one exemplary embodiment of the invention, the alignment of the probe relative to the first observation plane is tracked by a navigation system before the first determination of an angular position by the motion sensor.

Typically, an alignment of the probe with respect to the operation site can thus be determined beforehand and as a start value for a motion detection that follows by the motion sensor. The system can be calibrated by the navigation system after being switched-on, and an angular position and/or a position in space can be calculated.

In one exemplary embodiment of the invention, with an additional navigation system, a position and/or an alignment of the probe of the endoscope are/is determinable by tracking of a navigation element arranged on the endoscope.

A navigation system can already be part of the equipment of a surgical system or is additionally supplementable. Typically, this can be used to determine an absolute spatial position and/or angular position of the endoscope by a tracking element. The combination of navigation system and motion sensor enables the angular position of the endoscope to be determined very precisely. Typically, further surgical tools or the patient's body part to be operated on can be tracked by the navigation system.

In one exemplary embodiment of the invention, with an additional navigation system, an angular position of the probe of the endoscope is determinable by tracking of a navigation element arranged on the endoscope.

It may be sufficient to determine an angular position of the probe in space by a tracking element.

In one exemplary embodiment of the invention, the navigation system is formed by an electromagnetic tracking system having at least one transmitter and at least one receiver.

Electromagnetic tracking between the observation apparatus and the probe has the advantage over the conventional navigation solutions that no navigation elements, for example navigation image recording devices, having an adverse effect on visibility or handling need be mounted on the probe of the endoscope. By way of example, it would be necessary merely to accommodate an RFID chip or a solenoid in the handle of the endoscope or to mount it on the handle. Moreover, the distance from the observation apparatus, for example a surgical microscope or a camera, and the endoscope is in a favorable range for electromagnetic tracking.

In one exemplary embodiment of the invention, at least two different images captured by the second image recording device of the endoscope at two different points in time are represented on the display device.

The display of two different images allows the representation of preoperative image data together with current image data. Moreover, two views can be represented at two different points in time. Alternatively, the display of an individual image together with a video live image is conceivable.

In one exemplary embodiment of the invention, the first image of the observation apparatus and the second image of the endoscope are displayed in a “Picture-In-Picture” representation on the display device.

A “Picture-In-Picture” representation is the display of the second image as an inserted sub-picture in the first image. For this purpose, the second image can be represented with reduced size or be represented only partly in an excerpt. As a result of the spatial proximity of the first image and the second image, the images can be registered visually more rapidly by a user.

In one exemplary embodiment of the invention, a motion value is determinable by an analysis of the images provided by the second image recording device.

A second image recording device of the endoscope can record images in temporal succession. A motion value can be derived therefrom in the control unit, for example by image processing software. The image capture system thus forms an additional motion sensor that improves the motion detection and resolution of the overall system even further.

In one exemplary embodiment of the invention, the power supply of the endoscope is wire-free and comprises a battery or a rechargeable battery.

In the case of battery- or rechargeable-battery-operated medical apparatuses, it is possible to dispense with a connecting cable. As a result, the handling of the endoscope is simpler and more flexible since no cable need be carried along in the event of a change in the position of the endoscope.

In one exemplary embodiment of the invention, the observation apparatus is a surgical microscope.

Surgical microscopes can comprise image recording devices, for example image recording sensors or cameras. A digital surgical microscope can be formed by a camera having an optical unit. Typically, an endoscope can be retrofitted to supplement an already existing surgical microscope.

In one exemplary embodiment of the invention, the observation apparatus is a camera.

A camera is compact and cost-effective and scarcely impedes an observer during an examination or operation.

### BRIEF DESCRIPTION OF THE DRAWINGS

The invention will now be described with reference to the drawings wherein:

FIG. 1 shows an observation apparatus and an endoscope in an operation scenario;

FIG. 2 shows an enlarged excerpt from the operation scenario in accordance with FIG. 1 with a first coordinate system;

FIG. 3 shows a surgical microscope image together with an endoscope image;

FIG. 4 shows the microscope image and the endoscope image in a mutually aligned arrangement;

FIG. 5 shows the endoscope in accordance with FIG. 1 with a motion sensor and the insertion of a graphical marking on a display device;

FIG. 6 shows a display device with one example of a Picture-in-Picture arrangement of a plurality of endoscope images with a graphical marking depending on the alignment of the viewing direction of the probe of the endoscope;

FIG. 7 shows a surgical microscope and an endoscope in an operation scenario with electromagnetic tracking of the probe.

### DESCRIPTION OF EXEMPLARY EMBODIMENTS

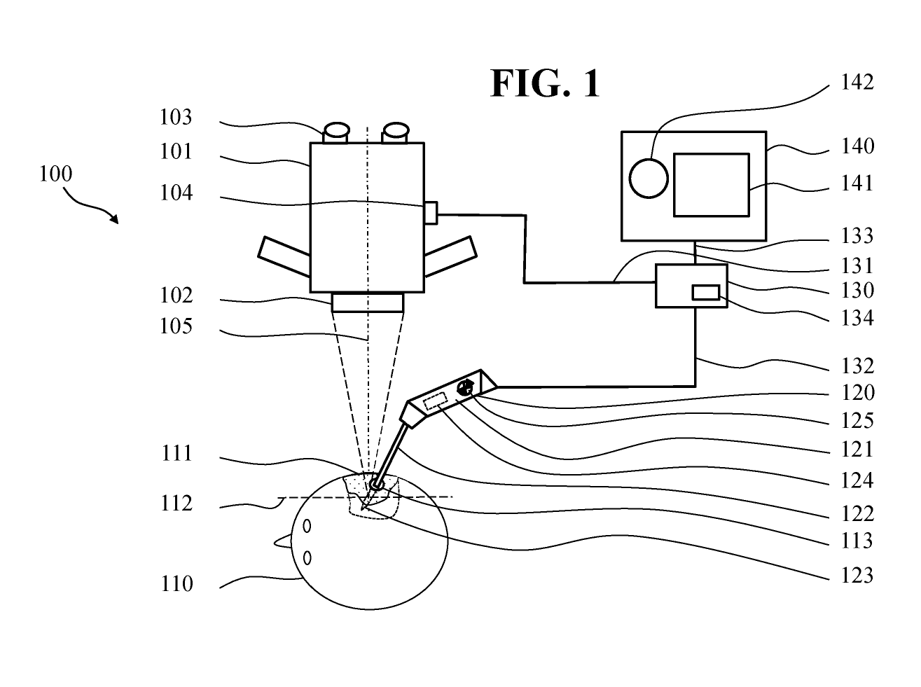

FIG. 1 shows an observation apparatus and an endoscope 120 in an operation scenario 100.

The observation apparatus is a surgical microscope 101. The surgical microscope 101 having a main objective 102 is represented for the observation of an object 110 to be observed, for example a patient's head. The main objective 102 has an optical axis 105. The surgical microscope is configured as a stereo microscope. An observer or surgeon can view an operation region 111 with an object plane, which is referred to as first observation plane 112, through the eyepieces 103. The surgical microscope 101 comprises a first image recording device 104. The image recording device 104 captures an image or a video sequence of the operation region 111.

The tissue to be operated on in the operation region 111 is additionally observed via the endoscope 120. The endoscope 120 comprises a handpiece 121 and a probe 122. The handpiece 121 is arranged in an angled manner relative to the probe; the angle is 45°, for example. Grip surfaces (not illustrated) can be mounted on the exterior of the handpiece 121. A second image recording device 124, depicted by dashed lines, a motion sensor 125, and also an illumination device (not illustrated) and an interface for data communication are arranged in the interior of the handpiece 121.

The probe 122 comprises a long thin tube having a probe tip 123. The probe tip 123 defines the distal end of the probe 122. The probe 122 is introduced into the tissue in the operation region 111 via a body opening 113 in order to view anatomical structures or body tissue behind the first observation plane 112. An optical unit (not illustrated) is arranged on the probe tip 123. The probe 122 comprises a first optical waveguide for illuminating a tissue region and a second optical waveguide, which is led from the optical unit on the probe tip 123 to the second image recording device 124. In one exemplary embodiment, the optical waveguide can also be formed by an electron conductor. In one exemplary embodiment, the image capture device can also be arranged on the probe tip 123.

The first image recording device 104 is connected to a control unit 130 via first line 131. The endoscope 120 is connected to the control unit 130 by a second line 132. The control unit 130 comprises an image processing unit 134. The control unit 130 is coupled to a display device 140 via a third line 133. The display device 140 shows the image captured by the first image recording device 104 of the surgical microscope 101 in a first image 141. The image captured by the second image recording device 124 of the endoscope 120 is represented in a second image 142 on the display device 140.

The images captured by the first image recording device 104 of the surgical microscope 101 or the second image recording device 124 of the endoscope 120 can in each case represent individual images or video sequences.

The surgical microscope 101 can be a conventional optical stereo surgical microscope, wherein the observation region can be viewed through the eyepieces 103. The surgical microscope 101 can also be configured as a purely digital surgical microscope, wherein the operation region 111 with the first observation plane 112 is recorded by the first image recording device 104 and represented on the display device 140. The surgical microscope 101 can also be configured as a hybrid system and both enable an observation through eyepieces 103 and have one or more first image recording devices 104 for representing the observation region with the first observation plane 112. The surgical microscope 101 can also be formed by a single camera. The first image 141 of the first image recording device 104 of the surgical microscope 101, said first image being represented on the display device 140, can be displayed as a two- or three-dimensional image.

The endoscope 120 can furthermore have an energy store for power supply independent of the electricity grid, for example a battery or a rechargeable battery or a capacitor having a very large capacitance. The endoscope 120 is hermetically encapsulated. The endoscope is fully autoclavable. In use during an operation, however, the endoscope 120 can also be protected by a sterile protective film, referred to as a drape.

The control unit 130 is formed by a microcontroller assembly or an industrial computer, for example. The image processing unit 134 is part of the control unit 130 and comprises a hardware and/or a software module. The control unit 130 can be integrated into the surgical microscope 101 or in the display device 140. The control unit 130 can also be divided into a plurality of assemblies. An assembly of the control unit 130 can be integrated into the endoscope 120. The first line 131, the second line 132 and the third line 133 can be formed in wired or wireless fashion. A wired line can be a network line or a data line, for example a coaxial cable or a fiber-optic cable. A wireless connection can be formed by radio, WLAN or Bluetooth and in each case comprise a transceiver unit.

The first image recording device 104 of the surgical microscope 101 or the second image recording device 124 of the endoscope 120 can be in each case a camera or an image sensor, for example a charge-coupled device (CCD) chip. An image recording device can record monochrome images and/or color images. An image recording device can also be configured to record fluorescence images. One or a plurality of optical elements (not illustrated), for example lenses, stops or filters, can be arranged upstream of the image sensor. An image recording device can comprise a single image sensor or a plurality of image sensors and can be configured to record 2D or 3D images. An endoscope 120 can also be an ultrasonic probe.

The display device 140 is a screen, which can be configured as a 2D screen or a 3D screen. In an exemplary embodiment, the display device 140 is a data projection device in the surgical microscope 101. A data projection device is a display device whose image is inserted into one or both observation beam paths of the surgical microscope 101. A data projection device can represent a monochrome image or a colored image. The data projection device can represent the image recorded by the second image recording device 124 of the endoscope 120 together with additional information. Additional information can be preoperative images or text information, for example. A 2D screen or a 3D screen can also be present together with the data projection device.

If the display device 140 is a screen, the images of the first image recording device 104 of the surgical microscope 101 and of the second image recording device 124 of the endoscope 120 can be displayed together. In this case, the second image 142, the endoscope image, can be represented as a sub-picture in the first image 141 captured by the surgical microscope. This is referred to as “Picture-in-Picture” representation.

In an exemplary embodiment, the first line 131 is led from the first image recording device 104 directly to the display unit 140. For this purpose, the first line 131 can also be led through the control unit 130, without being connected to the image processing unit 134. The control unit can comprise information about the alignment of the first viewing axis Y1. This information can be stored as a fixed value in the control device.

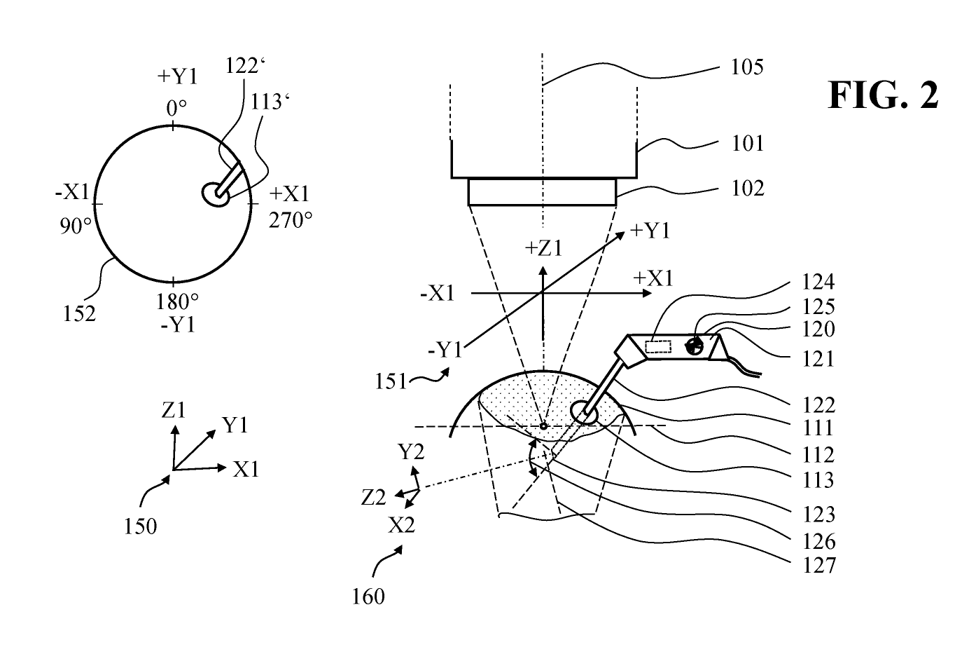

FIG. 2 shows an enlarged excerpt from the operation scenario in accordance with FIG. 1 with a first coordinate system 150.

The first coordinate system 150 comprises the orthogonal axes X1, Y1 and Z1. The first coordinate system 150 is additionally represented below the main objective 102, perpendicular to the optical axis 105, and is identified by the reference sign 151. Said first coordinate system is also defined for the first observation plane 112. The axis Z1 is formed by the optical axis 105. The observer (not illustrated) is situated at a position in front of the operation region 111 and looks from a direction −Y1 in the direction +Y1. This direction of view defines the first viewing direction of the observer relative to the surgical microscope. This first viewing direction is the “0°” viewing direction for the observer. The axis Y1 forms the first viewing axis. The X1-axis is defined orthogonally to the axis Y1. From the observer's viewpoint, the −X1-axis segment is defined as left, and the +X1-axis segment is defined as right.

A surgical microscope image 152 shows a representation of the image that can be viewed through the surgical microscope 101. The surgical microscope image 152 can be viewed through the eyepieces 103. In addition, the surgical microscope image 152 is recorded by the first image recording device 104 and can be displayed as a first image 141 on the display device 140, as shown in FIG. 1. The X1-axis runs from left to right. The axis Y1, defining the first viewing direction of the observer, runs from bottom to top. The first viewing direction “0°” defined for the observer is marked at the top in the surgical microscope image 152.

The surgical microscope image 152 shows the operation region 111 to be observed. Moreover, part of the probe 122 is visible, which is designated by the reference sign 122′.

The probe 122 is introduced into the tissue in the operation region 111 via the body opening 113, designated by the reference sign 113′. The probe tip 123 of the probe 122 is not visible in the surgical microscope image 152.

An optical unit, configured as a wide-angled optical unit, is arranged on the probe tip 123 of the endoscope 120, such that the direction of view of the probe tip 123 is not implemented in an extension of the center axis of the probe 122, but rather at an angle with respect to the center axis thereof. Said angle is approximately 45°, relative to the center axis of the probe 122. The wide-angle optical unit arranged on the probe tip 123 brings about an enlarged aperture angle 126. The aperture angle 126 of the wide-angle optical unit is 100° in this exemplary embodiment. In addition, the handpiece 121 is angled by a specific angle relative to the probe 122. Said angle is 45°, for example.

In an exemplary embodiment, the probe tip can also be configured in a different shape and have a different direction of view and a different aperture angle.

The second image recording device 124 of the endoscope 120 can record an image of anatomical structures below the first observation plane 112 from a lateral direction in a second observation plane 127. The second observation plane 127 differs from the first observation plane 112. The first observation plane 112 and the second observation plane 127 are arranged at an angle with respect to one another. Said angle is 80°, for example. The image recorded by the second image recording device 124 is referred to as endoscope image. The endoscope image defines a second coordinate system 160 having the orthogonal axes X2, Y2 and Z2.

The second viewing direction of the probe 122 is defined by the geometric and optical construction of the endoscope 120. In this exemplary embodiment, the second viewing direction of the probe 122 is defined by the Y2-axis. The Y2-axis lies in the plane spanned by the center axis (not illustrated) of the probe 112 and of the handpiece 125. The Y2-axis forms the second viewing axis.

In the endoscope image, the midpoint of the second observation plane 127 lies at the center of the observation cone spanned by the wide-angle optical unit. In FIG. 2, the midpoint of the endoscope image is marked as rearward extension of the Z2-axis of the second coordinate system 160. Therefore, the midpoint of the endoscope image does not lie in an extension of the center axis of the probe 122, where the observer would intuitively expect the midpoint. In the endoscope image, the region which lies in the extension of the center axis of the probe 122 is represented at the image edge, in the negative region of the Y2-axis, as it were in a 180° position.

For the observer who manually guides the endoscope 120, this angled configuration poses a certain challenge for hand-eye coordination. This is additionally made more difficult since the probe tip 123 in the operation channel lying in the tissue in the operation region 111 below the body opening 113 is not visible to the observer either with the naked eye or with the surgical microscope 101.

The anatomical structure to be viewed in the surgical microscope, for example an aneurysm, hides part of the probe 122 and the probe tip 123. Moreover, the probe tip 123 may be particularly close to tissue to be dealt with carefully or a structure to be dealt with carefully. An erroneous movement of the probe 122 in the axial direction of the center axis of the probe 122, deeper into the operation channel in the advance direction, might bring about undesired tissue damage.

Therefore, a graphical marking is inserted in the second image 142, the endoscope image, represented on the display device 140, said graphical marking indicating the direction of the second viewing axis Y2 in the second image.

In one exemplary embodiment, the second image 142 represented on the display device 140 is rotated in such a way that the second viewing axis Y2 corresponds to the first viewing axis Y1. In this exemplary embodiment, the second image 142 is rotated by an angle in such a way that the second viewing axis Y2 is arranged vertically. The image region lying in the Y2-direction is displayed at the top.

In one exemplary embodiment, the image rotation of the second image 142 is carried out together with the display of the graphical marking.

In another exemplary embodiment, the graphical marking can also mark an image region which displays a straight ahead view in the advance direction of the probe 122. In this exemplary embodiment, the advance direction lies in a 180° position, i.e., in the vicinity of the lower image edge of the second image 142.

All the variants mentioned above can be present individually or in combination. It is conceivable for two graphical markings to mark a viewing axis Y2 and an advance direction and additionally for the second image to be represented in a manner rotated by an angle on the display device 140.

It is also conceivable for the image rotation of the second image 142 to be carried out without a display of the graphical marking. By way of example, the second image 142 is rotated by an angle in such a way that the second viewing axis Y2 is arranged vertically. The image region lying in the Y2-direction is displayed at the top. In this exemplary embodiment, a display of the graphical marking can be dispensed with.

The rotation of the second image and/or a graphical marking make(s) possible for the observer a reliable orientation in the second image 142 represented on the display device 140 and an unambiguous assignment of the tissue region lying in the advance direction of the probe 122 and thus significantly facilitate(s) hand-eye coordination.

The surgical microscope image 152 shows a part of the probe 122′. The surgical microscope image 152 is evaluatable by the control unit 130. An alignment of the probe 122′ relative to the first observation plane 112 is thus determinable by evaluation of the image information of the first image recording device 104. This information about the alignment of the probe 122′ can supplement the items of information provided by the motion sensor 125 and/or can be used as a start value. The system can be calibrated on the basis of this information.

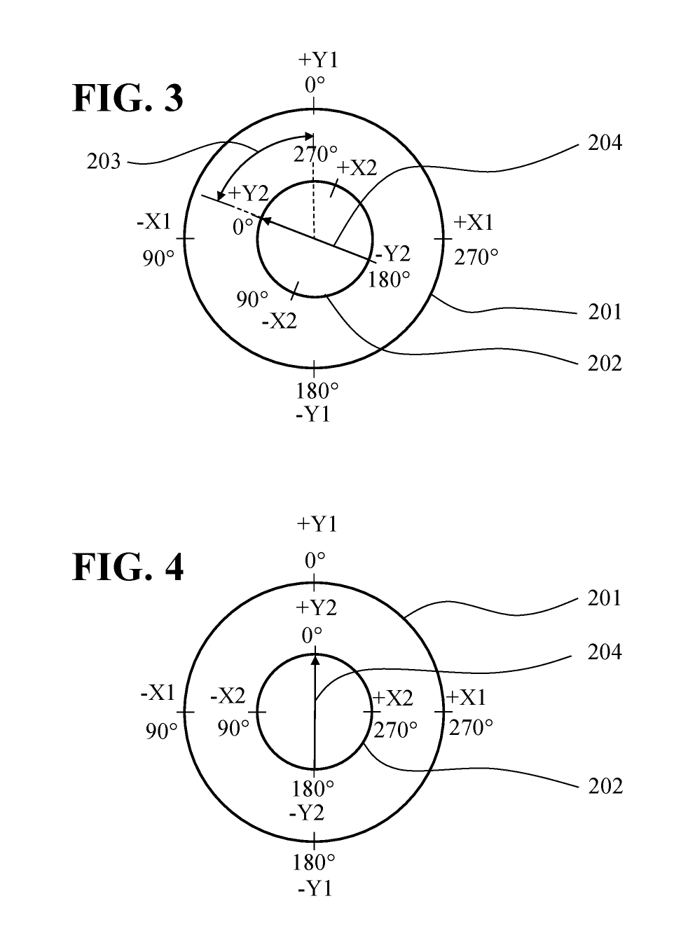

FIG. 3 shows a surgical microscope image 201 together with an endoscope image 202. For explanation purposes, the endoscope image 202 is arranged at the center of the surgical microscope image 201. The surgical microscope image 201 in accordance with FIG. 3 corresponds to the surgical microscope image 152 in accordance with FIG. 2.

The first viewing direction of the observer relative to the first observation plane 112 is defined by the first viewing axis Y1. The second viewing direction of the endoscope is defined by the second viewing axis Y2.

The surgical microscope image 201 shows, in the Y1-direction or in the “0°” position, the first viewing direction toward the operation region 111, in a manner such as the latter can be viewed by the observer even without a surgical microscope in the first viewing direction along the first viewing axis Y1. The observer designates this “0°” position as “top”.

By contrast, the endoscope image 202 is rotated by the angle 203. The second viewing axis Y2 of the endoscope image 202, which second viewing axis would be designated as “top” by the observer on account of the holding position of the endoscope, is thus arranged in a manner rotated by the angle 203, for example 70°, relative to the first viewing axis Y1 of the surgical microscope image 201.

Upon a rotation of the endoscope about the axis of the probe or upon a movement of the probe in the advance direction, i.e., in the axial direction of the axis of the probe, the represented image excerpt and/or the angle 203 of the endoscope image 202 change(s). Without information about said angle 203, the hand-eye coordination of the observer who is manually guiding the endoscope is hampered. This leads to vexation during movement of the endoscope and during assignment of the image contents.

Therefore, a graphical marking 204 is inserted in the represented second image, the endoscope image 202, said graphical marking indicating the direction of the second viewing axis Y2 in the second image. This graphical marking 204 is configured as a line with a direction arrow indicating the position and direction of the second viewing axis Y2. The observer can thus recognize very simply the relative orientation of the endoscope image 202 with respect to the viewing axis of the surgical microscope image 101. This facilitates guidance of the endoscope and hand-eye coordination for the observer.

The graphical marking 204 can be embodied in various geometric shapes and/or colors. The graphical marking 204 can be configured for example as a single arrow or a single line, a pin, a triangle or a line at the image edge. The graphical marking can be arranged at the upper or lower image edge or offset from the image edge, at the image center or at an arbitrary location in the image. The graphical marking 204 can be embodied in various suitable colors that contrast well in terms of color with the tissue being viewed, e.g., green or yellow. The colors can be freely selectable or fixedly preset. Even the exemplary embodiment as a short line segment at the image edge, along the second viewing axis Y2, may be sufficient. The line segment can have for example a length having an absolute value in a range of between 3% and 10% of the diameter of the endoscope image 202.

FIG. 4 shows the microscope image 201 and the endoscope image 202 in accordance with FIG. 3 in a mutually aligned arrangement.

The endoscope image 202 is arranged in a manner rotated in the clockwise direction by the angle 203, which is 70° in this example, relative to the endoscope image 201, such that the second viewing axis Y2 of the endoscope image 202 corresponds to the first viewing axis Y1 of the microscope image 201.

The second orientation of the second image, the endoscope image 201, is thus aligned relatively to the first viewing axis Y1 depending on the angular position of the probe, the angle 203. As a result of the rotation of the endoscope image 202, the viewing and working direction of the endoscope now corresponds to that of the surgical microscope.

Since the motion sensor captures an angular position and/or angular change, which the control unit processes and evaluates, the alignment of the graphical marking 204 in the second image can be automatically tracked. This facilitates the hand-eye coordination of the observer holding the endoscope by hand and improves the handling of the endoscope.

In one exemplary embodiment, the first image recording device 104 is directly connected to the display device 140. In this case, the control unit 130 is connected only to the second image recording device 124 and the display device. Information about the orientation of the first viewing axis Y1 is stored in the control unit 130, such that the orientation of the second image is alignable relative to the viewing axis Y1 and/or the graphical marking 204 in the second image is alignable. The orientation of the second image and/or the graphical marking 204 are/is trackable depending on an angular position of the probe 122 relative to the first viewing axis Y1.

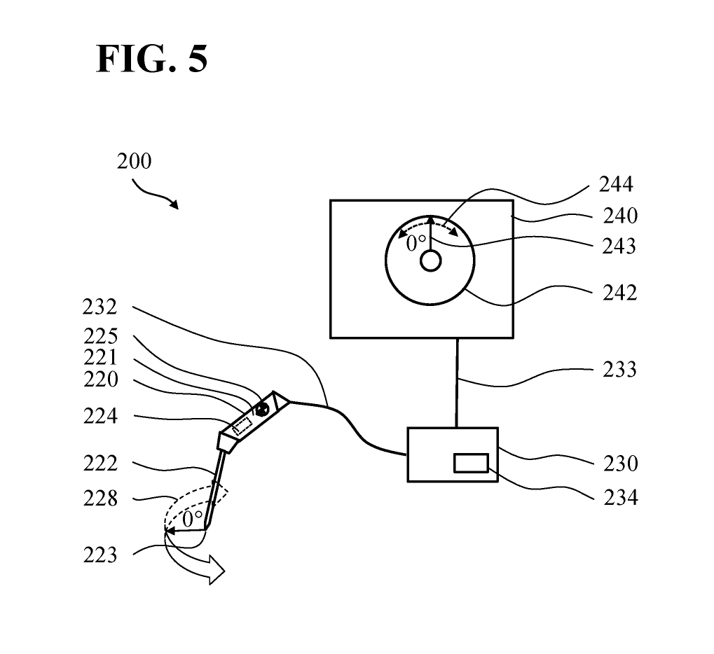

FIG. 5 shows the endoscope in accordance with FIG. 1 with a motion sensor and the insertion of a graphical marking on a display device.

The visualization system 200 has the same components as the visualization system in the operation scenario 100 in accordance with FIG. 1, with the reference signs being increased by 100. The illustration in FIG. 5 differs from the illustration in accordance with FIG. 1 in that it shows an endoscope 220 with a control unit 230 and a display device 240 without a surgical microscope.

The endoscope 220 comprises a probe 222 having a probe tip 223, a second image recording device 224, illustrated by dashed lines, and a motion sensor 225. The endoscope 220 is connected to the control unit 230 by a second line 232. The control unit 230 is coupled to the display device 240 via a third line 233. The control unit 230 comprises an image processing unit 234. The image recorded by the second image recording device 224 of the endoscope 220 is represented in a second image 242 on the display device 240. A graphical marking 243 indicates the second viewing direction Y2, or the “0°” position, of the endoscope 220. The graphical marking 243 is superimposed or inserted into the image communicated by the image recording device 224 by means of the image processing unit 234.

Upon a rotation of the endoscope 220 about the center axis of the probe 222 toward the right or left, the viewing direction, or the “0°” position, of the endoscope 220 likewise changes toward the right or left. This rotational movement is represented by the semicircular first double-headed arrow 228. An angular change during this rotational movement is detected by the motion sensor 225 and communicated to the control unit 230. As a result, it is possible to calculate anew the representation of the second image 242, depending on the angular change of the endoscope 220, relative to the first viewing axis of the surgical microscope and to track the position of the graphical marking 243 anew in each case. The second image 242 represented by the image recording device 224 shows the viewing direction of the endoscope 220 and can be displayed together with the graphical marking 243 in two ways.

In a first representation variant, the second image 242 is displayed relative to the first viewing direction of the surgical microscope in such a way that the second viewing axis Y2 of the endoscope 220 corresponds to the first viewing axis Y1 of the microscope. In this case, the graphical marking 243 points in the same direction as the first viewing axis of the surgical microscope, for example upward.

In a second representation variant, the second image 242 is displayed at a rotation angle relative to the first viewing direction of the surgical microscope, wherein the graphical marking 243 indicates the second viewing axis Y2 of the endoscope 220 relative to the first viewing axis Y1 of the surgical microscope. The graphical marking 243, representing the viewing direction, or the “0°” position, of the endoscope 220, is carried along synchronously with a rotational movement of the probe 222 of the endoscope 220 on the display device 240. This is illustrated by the second double-headed arrow 244.

In this way, an orientation relative to the images displayed on the display device 240 is possible very simply for the observer.

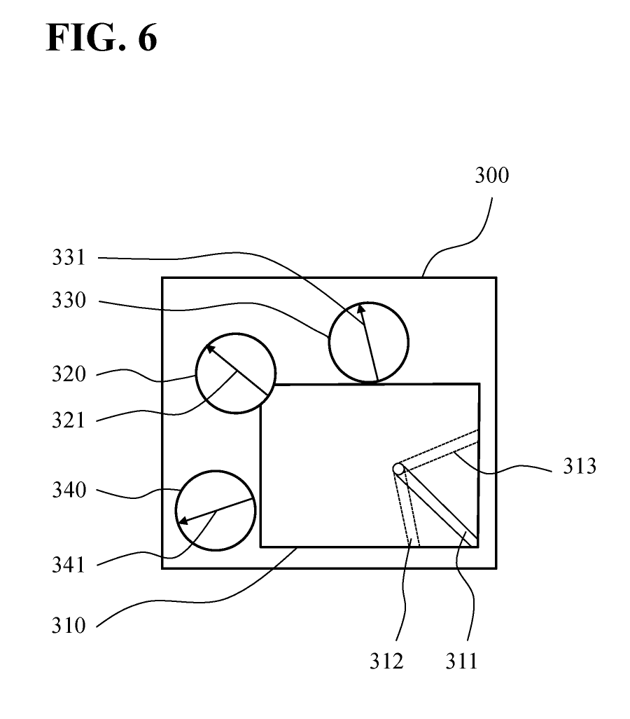

FIG. 6 shows a display device 300 with one example of a picture-in-picture arrangement of a plurality of endoscope images with a graphical marking depending on the alignment of the viewing direction of the probe of the endoscope.

The display device 300 shows a surgical microscope image, for example the representation of an operation site, in a rectangular first image 310. A first position of the probe 311 of an endoscope at a first point in time is visible in the surgical microscope image. The associated endoscope image at said first point in time is represented in a round second image 320. A second viewing axis of the endoscope, relative to the first viewing axis of the surgical microscope, is indicated by a first graphical marking 221.

An angular change to a second position of the probe 312 at a second point in time is captured by the motion sensor in the endoscope. The image captured at the second point in time is displayed in a round third image 330. A second graphical marking 331 shows the second viewing axis of the endoscope relative to the first viewing axis of the surgical microscope at said second point in time.

A further angular change to a third position of the probe 313 at a third point in time is captured by the motion sensor in the endoscope. The image captured at a third point in time is displayed in a round fourth image 340. A third graphical marking 341 shows the second viewing axis of the endoscope relative to the first viewing axis of the surgical microscope at said third point in time.

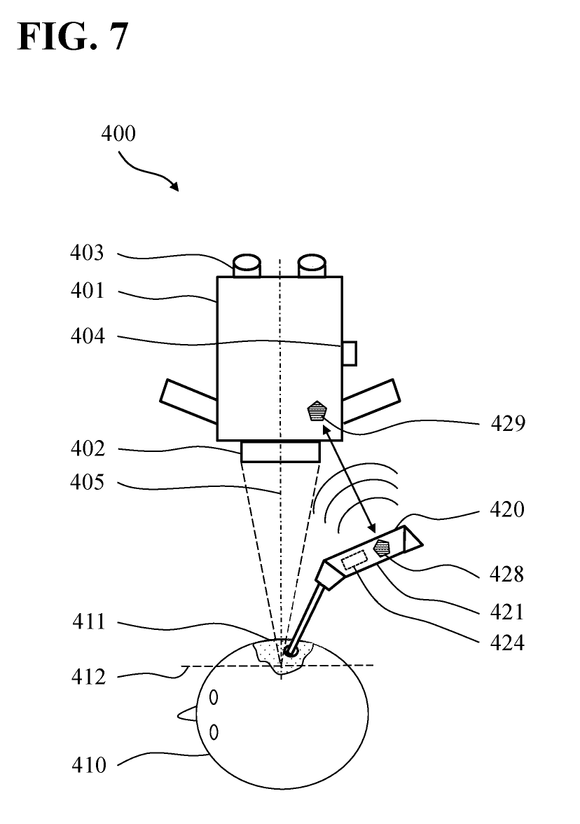

FIG. 7 shows a surgical microscope and an endoscope in an operation scenario 400 with electromagnetic tracking of the probe.

The operation scenario 400 has a visualization system having the same components as the visualization system in the operation scenario 100 in accordance with FIG. 1, with the reference signs being increased by 300.

An endoscope 420 in accordance with FIG. 7 differs from the endoscope 120 in accordance with FIG. 1 in that the motion sensor 125 is replaced by a first electromagnetic tracking element 428.

The first electromagnetic tracking element 428 is related to a second electromagnetic tracking element 429 arranged on a surgical microscope 401. The first electromagnetic tracking element 428 and the second electromagnetic tracking element 429 can be formed by a transceiver pair. For this purpose, by way of example, an RFID chip or a solenoid can be arranged in a handpiece 421 of the endoscope. The distance between the handpiece 421 of the endoscope 420 and the surgical microscope 401 is in a favorable range for electromagnetic tracking. An arrangement of the first electromagnetic tracking element 428 within the handpiece 421 has the advantage that no outer tracking elements are arranged on the endoscope 420, which outer tracking elements would hamper handling or have a disadvantageous effect on the view of the operation region 411. It is also conceivable for the first tracking element 428 and the second tracking element 429 to be detectable by an additional navigation system (not illustrated).

In another exemplary embodiment, both a first tracking element 428 and a motion sensor (not illustrated), for example a position or acceleration sensor, are arranged in the handpiece 421 of the endoscope 420. The combination of electromagnetic tracking and a motion sensor enables a very accurate motion and position detection of the endoscope 420.

In an exemplary embodiment of the invention in accordance with FIGS. 1 to 7, the visualization system comprises a first observation apparatus having a first image recording device 104, 404 for observing an operation region 111, 411 with a first observation plane 112, 412, wherein in the first observation plane 112, 412 a viewing direction is defined by a first viewing axis Y1, and an endoscope 120, 220, 420 having a probe 122, 122′, 222 and a second image recording device 124, 224, 424 for observing the operation region 111, 411 with a second observation plane 127 with a second viewing axis Y2.

The visualization system comprises a display device 140, 240, 300, which represents a first image 141, 310 recorded by the first image recording device 104, 404 in a first orientation and a second image 142, 242, 320, 330, 340 recorded by the second image recording device 124, 224, 424 in a second orientation, and a control unit 130, 230, which is connected to the first image recording device 104, 404, the second image recording device 124, 224, 424 and the display device 140, 240, 300.

The endoscope 120, 220, 420 comprises a motion sensor 125, 225, which is connected to the control unit 130, 230, an angular position of the probe 122, 122′, 222 of the endoscope 120, 220, 420 in space being determinable by said motion sensor, where the control unit 130, 230 is configured to the effect that an angular position of the probe 122, 122′, 222 of the endoscope 120, 220, 420 relative to the first viewing axis Y1 is determinable by evaluation of the data of the motion sensor 125, 225, such that the second orientation of the second image 142, 242, 320, 330, 340 is alignable depending on an angular position of the probe 122, 122′, 222 relative to the first viewing axis Y1.

In one exemplary embodiment, a graphical marking 204, 321, 331, 341 is inserted in the second image 142, 242, 320, 330, 340 represented on the display device 140, 240, 300, said graphical marking indicating the direction of the second viewing axis Y2 in the second image 142, 242, 320, 330, 340, wherein the graphical marking 204, 321, 331, 341 is trackable depending on an angular position of the probe 122, 122′, 222 relative to the first viewing axis Y1.

In one exemplary embodiment, the first observation apparatus is a surgical microscope 101, 401. The surgical microscope 101, 401 can be a conventional surgical microscope having eyepieces and at least one camera, or a purely digital, camera-based, surgical microscope.

In one exemplary embodiment, the first observation apparatus is a camera. The camera can be a commercially available camera or a camera with an additional optical unit.

According to a further exemplary embodiment of the invention, the endoscope can also be some other image capture device, for example a manually guided camera or an image capture device that can capture images on the basis of ultrasound.

The term “comprising” (and its grammatical variations) as used herein is used in the inclusive meaning of “having” or “including” and not in the exclusive sense of “consisting only of.” The terms “a” and “the” as used herein are understood to encompass the plural as well as the singular.

It is understood that the foregoing description is that of the exemplary embodiments of the invention and that various changes and modifications may be made thereto without departing from the spirit and scope of the invention as defined in the appended claims.

### LIST OF REFERENCE NUMERALS

100, 400 Operation scenario
101, 401 Surgical microscope
102, 402 Main objective
103, 403 Eyepieces
104, 404 First image recording device
105, 405 Optical axis
110, 410 Object to be observed
111, 411 Operation region
112, 412 First observation plane
113, 113′ Body opening
120, 220, 420 Endoscope
121, 421 Handpiece
122, 122′, 222 Probe
123, 223 Probe tip
124, 224, 424 Second image recording device
125, 225 Motion sensor
126 Aperture angle
127 Second observation plane
130, 230 Control unit
131 First line
132, 232 Second line
133, 233 Third line
134, 234 Image processing unit
140, 240 Display device
141 First image
142, 242 Second image
150 First coordinate system
151 First coordinate system
152 Surgical microscope image
160 Second coordinate system
200 Visualization system
201 Surgical microscope image
202 Endoscope image
203 Angle
204 Graphical marking
228 First double-headed arrow
243 Graphical marking
244 Second double-headed arrow
300 Display device
310 First image
311 First position of the probe
312 Second position of the probe
313 Third position of the probe
320 Second image
321 First graphical marking
330 Third image
331 Second graphical marking
340 Fourth image
341 Third graphical marking
428 First electromagnetic tracking element
429 Second electromagnetic tracking element

What is claimed is:

1. A visualization system comprising:
an observation apparatus having first image recording device configured to observe an operation region with a first observation plane, wherein in the first observation plane a viewing direction is defined by a first viewing axis Y1;
an endoscope having a probe and a second image recording device configured to observe the operation region with a second observation plane with a second viewing axis Y2;
a display device representing a first image recorded by the first image recording device in a first orientation and a second image recorded by the second image recording device in a second orientation;
a control unit connected to the second image recording device and to the display device,
wherein the endoscope comprises a motion sensor connected to the control unit, an angular position of the probe of the endoscope in space being determinable by said motion sensor, and
wherein the control unit is configured to permit the angular position of the probe of the endoscope relative to the first viewing axis Y1 to be determined by evaluation of data of the motion sensor and to permit the second orientation of the second image to be alignable depending on the angular position of the probe of the endoscope relative to the first viewing axis Y1.

2. The visualization system according to claim 1, wherein:
a graphical marking is inserted in the second image represented on the display device, said graphical marking indicating a direction of the second viewing axis Y2 in the second image,
the graphical marking is trackable in the second image depending on the angular position of the probe of the endoscope relative to the first viewing axis Y1.

3. The visualization system according to claim 1, wherein the control unit is connected to the first image recording device.

4. The visualization system according to claim 1, wherein the motion sensor is a sensor selected from a position sensor, an acceleration sensor, a vibration gyroscope sensor, and a gyrosensor.

5. The visualization system according to claim 1, wherein the second image recording device is fixedly connected to the probe.

6. The visualization system according to claim 1, wherein the second image recording device is arranged rotatably relative to the probe.

7. The visualization system according to claim 1, wherein an alignment of the probe relative to the first observation plane is determinable by image evaluation of images captured by the first image recording device.

8. The visualization system according to claim 1, wherein an alignment of the probe relative to the first observation plane is tracked by a navigation system before a first determination of the angular position by the motion sensor.

9. The visualization system according to claim 1,wherein with an additional navigation system, at least one of a position and an alignment of the probe of the endoscope is determinable by tracking of a navigation element arranged on the endoscope.

10. The visualization system according to claim 7, wherein a navigation system is formed by an electromagnetic tracking system having at least one transmitter and at least one receiver.

11. The visualization system according to claim 8, wherein the navigation system is formed by an electromagnetic tracking system having at least one transmitter and at least one receiver.

12. The visualization system according to claim 1, wherein at least two different images captured by the second image recording device of the endoscope at two different points in time are represented on the display device.

13. The visualization system according to claim 1, wherein the observation apparatus is a surgical microscope.

14. The visualization system according to claim 1, wherein the observation apparatus is a camera.
